(19) United States
(12) Patent Application Publication
(10) Pub. No.: US 2021/0100632 A1
(43) Pub. Date: Apr. 8, 2021

(54) COMPUTER-ASSISTED METHOD FOR PERFORMING SURGERY RELATIVE TO A PELVIS

(71) Applicant: ORTHOSOFT ULC, Montreal (CA)

(72) Inventors: Francois PARADIS, Boucherville (CA); Joel ZUHARS, Livermore, CA (US); Karine DUVAL, Montreal (CA); Mathieu CHEVRIER, Roxboro (CA); Louis-Philippe AMIOT, Montreal (CA); Herbert JANSEN, Freiburg (DE); Myriam VALIN, Laval (CA); Don DYE, Warsaw, IN (US); Isabelle ROBITAILLE, St-Ignace-de-Loyola (CA); Simon FERRON-FORGET, Deux-Montagnes (CA); Francois Baudoin, Montreal (CA)

(21) Appl. No.: 17/126,090

(22) Filed: Dec. 18, 2020

Related U.S. Application Data
(63) Continuation of application No. 13/954,279, filed on Jul. 30, 2013, now abandoned.
(60) Provisional application No. 61/677,106, filed on Jul. 30, 2012.

(51) Int. Cl. A61B 90/00; A61B 34/20

(52) U.S. Cl. CPC A61B 90/06; A61B 17/1742; A61B 34/20

(57) ABSTRACT

A method for performing a surgical procedure related to a pelvis of a patient comprises adjusting a length between ends of a digitizer device to a distance between opposite landmarks of the pelvis of the patient. The ends of the digitizer device are applied against the opposite landmarks of the pelvis. An inertial sensor unit of the digitizer device is initialized to set an orientation of the digitizer device relative to a medio-lateral axis of the patient, the medio-lateral axis of the patient being part of a pelvic coordinate system. A tool is navigated within the pelvic coordinate system during a surgical procedure.

### CROSS-REFERENCE TO RELATED APPLICATION(S)

The present application is a continuation of application Ser. No. 13/954,279 filed Jul. 30, 2013, which claims priority on U.S. Provisional Patent Application No. 61/677,106, filed on Jul. 30, 2012, the entire contents of both of which are incorporated by reference herein.

### FIELD OF THE APPLICATION

The present application relates to computer-assisted surgery using inertial sensors, for instance orthopedic surgery.

### BACKGROUND OF THE ART

Inertial sensors (e.g., accelerometers, gyroscopes, inclinometers, etc) are increasingly used in computer-assisted surgery for numerous reasons. Off-the-shelf inertial sensors are relatively inexpensive and produce results of sufficient precision and accuracy for orthopedic surgery applications.

A common trait of inertial sensors is that they often do not provide positional information but, rather, simply orientational information, as they operate relative to gravity. Therefore, methods must be devised to create bone references and tools considering the absence of positional information.

### SUMMARY OF THE APPLICATION

It is therefore an aim of the present invention to provide a novel method and system for creating a frame of reference for bones in computer-assisted surgery with inertial sensors.

Therefore, in accordance with a first embodiment of the present disclosure, there is provided a digitizer device comprising: an elongated body; legs connected to the elongated body; at least one joint between the legs and the elongated body such that free ends of the legs are displaceable relative to one another; and an inertial sensor unit connected to the elongated body, the inertial sensor unit having a preset orientation aligned with the elongated body.

Further in accordance with the first embodiment, the at least one joint comprises a translational joint in the elongated body.

Still further in accordance with the first embodiment, the translational joint is a telescopic joint between members of the elongated body.

Still further in accordance with the first embodiment, a locking device is on the translational joint to manually lock the joint.

Still further in accordance with the first embodiment, a receptacle is in the elongated body for releasably receiving the inertial sensor unit in such a way that the preset orientation of the inertial sensor unit is aligned with the elongated body.

Still further in accordance with the first embodiment, the free ends of the legs are pointy shaped.

Still further in accordance with the first embodiment, the at least one joint comprises translational joints on each said leg, to adjust a distance between the free ends and the elongated body.

Still further in accordance with the first embodiment, the preset orientation of the inertial sensor unit has an axis parallel to the legs.

Still further in accordance with the first embodiment, the preset orientation of the inertial sensor unit has an axis parallel to the elongated body.

In accordance with a second embodiment of the present disclosure, there is provided an assembly of a digitizer device and table reference device comprising: the digitizer device comprising: an elongated body; legs connected to the elongated body; at least one joint between the legs and the elongated body such that free ends of the legs are displaceable relative to one another; and an inertial sensor unit connected to the elongated body, the inertial sensor unit having a preset orientation aligned with the elongated body; the table reference device comprising: a body adapted to be fixed to an operating table; and an inertial sensor unit with a preset orientation related to the operating table; a patient coordinate system comprising orientation data obtained from the inertial sensor units of the digitizer device and the table reference device.

Further in accordance with the second embodiment, a receptacle is in the body of the table reference device for releasably receiving the inertial sensor unit in such a way that the preset orientation of the inertial sensor unit of the table reference device is aligned with a plane of the receptacle.

Still further in accordance with the second embodiment, the body of the table reference device comprises a bracket and hook for attachment to a rail of the operating table.

Still further in accordance with the second embodiment, the preset orientation of the inertial sensor unit in the table reference device has an axis normal to plane of the table.

In accordance with a third embodiment of the present disclosure, there is provided a method for creating at least part of a pelvic coordinate system of a patient in supine decubitus, comprising: adjusting a length between ends of a digitizer device to a distance between opposite landmarks of a pelvis of the patient; applying the ends of the digitizer device against the opposite landmarks of the pelvis; and initializing an inertial sensor unit of the digitizer device to set an orientation of the digitizer device relative to a medio-lateral axis of the patient, whereby the medio-lateral axis of the patient is part of the pelvic coordinate system.

Further in accordance with the third embodiment, a table reference device is positioned on an operating table supporting the patient in supine decubitus, and initializing an inertial sensor unit of the table reference device to set an orientation of the table reference device relative to a support plane of the table.

Still further in accordance with the third embodiment, a normal to the support plane of the table of the inertial sensor unit of the table reference device is set as an anterior-posterior axis of the patient in supine decubitus, whereby the anterior-posterior axis of the patient is part of the pelvic coordinate system.

### BRIEF DESCRIPTION OF THE DRAWINGS

FIG. 1 is a perspective view of a caliper instrument of a bone digitizer of the present disclosure;

FIG. 2 is a block diagram of the pelvic digitizer as part of a bone digitizing system of the present disclosure;

FIG. 3 is a flowchart of a method for creating a pelvic frame of reference with inertial sensors for subsequent tool navigation; and

FIGS. 4A-4C are perspective views of a table reference locator in accordance with an embodiment of the present disclosure.

### DESCRIPTION OF THE EXEMPLARY EMBODIMENTS

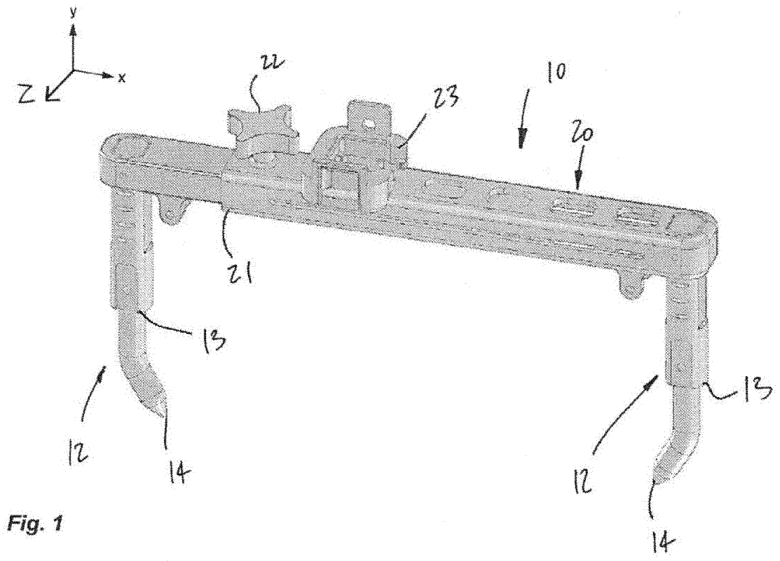

Referring to the drawings, and more particularly to FIG. 1, there is illustrated a caliper instrument 10 in accordance with the present application. The caliper instrument 10 may be used as part of a bone digitizer in a bone digitizing system, to create a frame of reference for subsequent navigation of tools relative to bones in surgery. The instrument 10 is referred to as a caliper, as it features a pair of legs 12 movable relative to one another, e.g., in a telescopic manner. The expression “caliper” is used nonrestrictively. Any other appropriate expression may be used to describe the instrument 10.

In the illustrated embodiment, the legs 12 of FIG. 1 each comprise a translational joint 13 so as to be expandable or contractible along the Y axis. For instance, the translational joints 13 may be any of sliding joint, telescopic joint, prismatic joint, indexing joint, etc. As an alternative, a single one of the legs may have a joint. It is also considered to use rotational joints as an alternative to translational joints 13, with an axis of the rotational joint being normal to a plane of the caliper instrument 10. A locking mechanism is typically provided, although not shown, to lock the translational joints 13 and, therefore, set the legs 12 in a selected length. The free end of each leg 12 has a pointy shape 14, although any other appropriate shape is considered, such as flat contact surfaces, discs, various concavities or convexities, etc., as a function of the type of bone or bodily part the caliper instrument 10 will be contacting. The pointy ends 14 of FIG. 1 are well suited to be used with a pelvis, with the pointy ends 14 contacting the anterior superior iliac spines (ASIS) on opposite sides of the pelvis, in pelvic surgery, with the patient in supine decubitus. Alternatively, the caliper instrument 10 could be used for the posterior superior iliac spine as well, or with other landmarks if the patient is in lateral decubitus.

Still referring to FIG. 1, the legs 12 are inter-connected by an elongated body 20 of the caliper instrument 10. The elongated body 20 features a translational joint 21 such that the elongated body 20 is expandable or contractible along the X axis. The translational joint 21 may be any appropriate joint, such as translational joints, telescopic joint, prismatic joints and/or indexing joints. It is also considered to use rotational joints as an alternative to the translational joint 21.

A locking device is generally shown at 22, and is of the type having a manual knob used to set the translational joint 21 in at a selected length, thereby allowing the user to set the length of the elongated body 20. An inertial sensor support or receptacle 23 is defined on the elongated body 20. The inertial sensor support 23 is, for instance, made with a specific geometry in order to precisely and accurately accommodate an inertial sensor unit in a predetermined complementary connection, simplifying a calibration between inertial sensor unit and caliper instrument 10. For instance, the inertial sensor unit has a preset orientation that is aligned with a dimension of the caliper instrument 10. In other words, the mechanical constraints in the attachment of inertial sensor unit 31 in the support 23 are such that the three axes of the inertial sensor unit 31 are aligned with the X, Y and Z axis of the caliper instrument 10. Therefore, the caliper instrument illustrated in FIG. 1 may expand and contract along both the X axis and the Y axis.

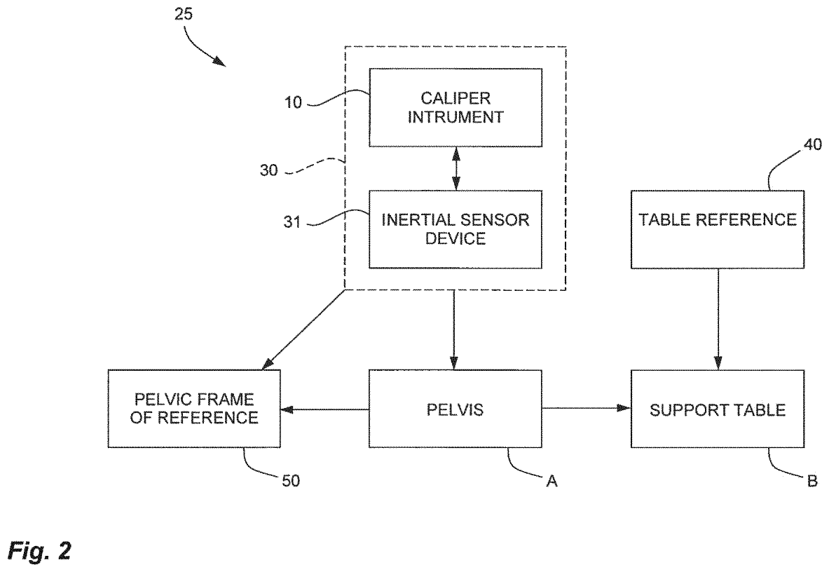

Referring to FIG. 2, the caliper instrument 10 is used as an instrument of a bone digitizing system 25, and is part of a bone digitizer 30 that features inertial sensor unit 31. The inertial sensor unit 31 may have any appropriate type of inertial sensor, to provide 3-axis orientation tracking. For instance, the inertial sensor unit 31 may have sets of accelerometers and/or gyroscopes, etc. The inertial sensor unit may be known as a sourceless sensor unit, as a micro-electromechanical sensor unit, etc. As mentioned above, the inertial sensor unit 31 is matingly received in the inertial sensor support 23 in a predetermined complementary connection, such that the initializing of the inertial sensor unit 31 will have the inertial sensor unit 31 specifically oriented relative to the X-Y-Z coordinate system illustrated in FIG. 1 (with the Z axis being the cross-product of the X and Y axes).

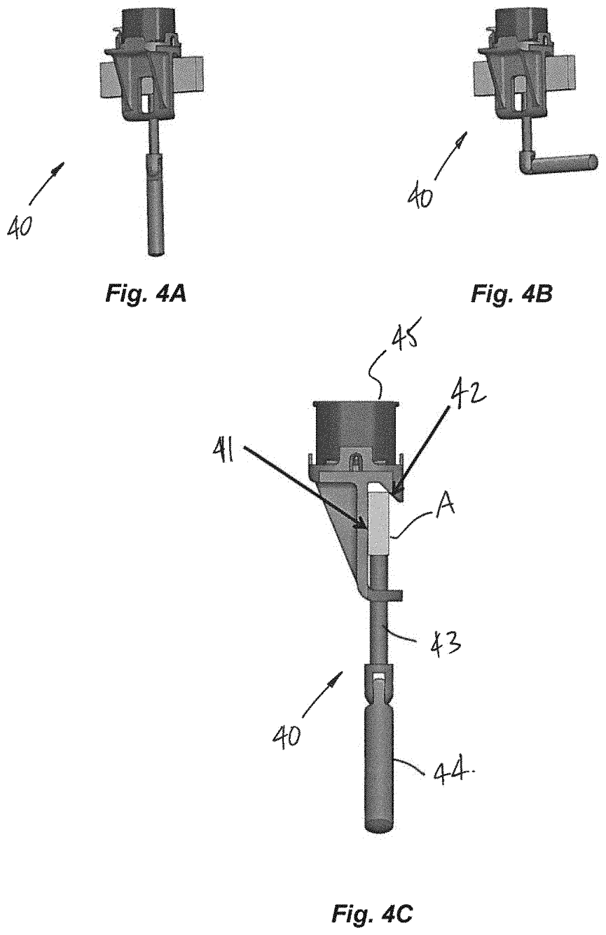

Still referring to FIG. 2, the bone digitizing system 25 may also comprise a table reference 40. Referring to FIGS. 4A, 4B and 4C, the table reference 40 is of the type comprising a body for planar engagement with the table plane and a flat surface for planar engagement with a lateral side of the table. In FIGS. 4A-4C, the table reference 40 has a body configured to attach to a rail of the table, with a bracket 41 accommodating the rail A in a lateral coplanar connection. A hook-like portion 42 faces the bracket 41 and hooks onto a top edge surface of the rail A. In order to fix the table reference 40 to the rail A, a bolt 43 may be screwingly engaged to a bottom of the bracket 41, with a pivotable handle 44 by which the bolt 43 may be tightened to block the table reference 40 against the rail A, in the manner shown in FIGS. 4A-4C, with the bracket 41 having its main surface parallel to that of the rail A. This configuration is one of numerous arrangements the table reference 40 may take.

The table reference 40 may comprise an inertial sensor unit 45 to produce a normal to the table plane and a normal to the table side (resulting in a table lateral axis). Accordingly, the table reference 40 is used to find a plane of support table B upon which the patient lies.

The table reference 40 may be combined with the optional bone digitizer 30, to determine the coordinate system of the pelvis A, in the pelvic application. Accordingly, the bone digitizing system 25 used in a pelvic application produces a pelvic frame of reference 50 for the subsequent navigation of tools relative to the pelvis A. The frame of reference 50 may be attached to a trackable reference (e.g., with 3-axis inertial sensors) in a secured relation relative to the bone.

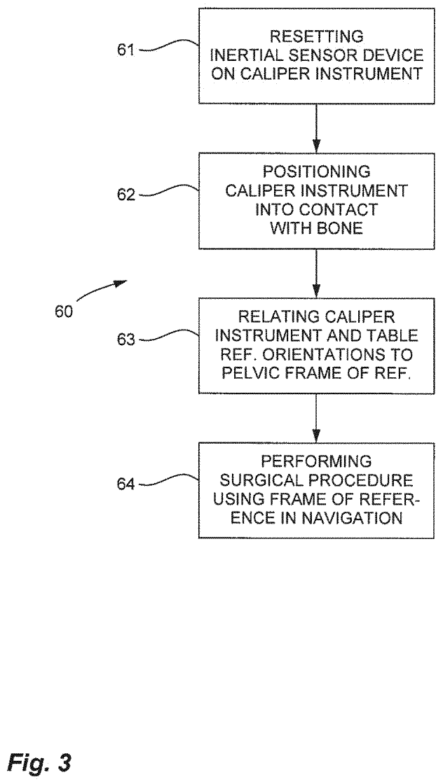

Now that the various components of FIGS. 1 and 2 have been described, a method for creating a frame of reference using inertial sensors for subsequent tool navigation is described in further detail with reference to FIG. 3, and is generally shown as 60.

According to 61, the inertial sensor unit 31 is reset once installed in the support caliper instrument 10. According to the embodiment of FIGS. 1 and 2, the resetting is facilitated by the complementary connection of the inertial sensor unit 31 in the inertial sensor support 23. According to an embodiment, the calibration is such that the X-Y-Z axes illustrated in FIG. 1 correspond to a 3-axis coordinate system of the inertial sensor unit 31. Accordingly, once the inertial sensor unit 31 is reset, an orientation of the caliper instrument 10 is known, for instance along the longitudinal axis of the caliper instrument 10, shown as the X-axis in FIG. 1.

According to 62, the caliper instrument 10 is positioned into contact with the bone. When the method 60 is used with the pelvis, the length of the caliper instrument in the X direction is set for the pointy ends 14 to be in contact with landmarks of the bone. When the patient is in supine decubitus or lateral decubitus, the landmarks may be the anterior (or posterior) superior iliac spines on both sides of the pelvis. As a result, a mediolateral (ML) axis of the pelvis may be set in the inertial sensor unit 31 when the caliper instrument 10 is in contact with the anterior superior iliac spines, with the legs 12 being arranged to be of the same height (in supine decubitus) or parallel to the table plane normal (in lateral decubitus).

According to 63, it may be desired to relate the table reference 40 to a reference orientation. For instance, the patient in supine decubitus lies on the support table B, and the plane normal of the support table B is used to define an antero-posterior axis of the pelvis, if the patient is in a strict supine decubitus, or quasi-strict supine decubitus. Accordingly, as shown in FIG. 2, the table reference 40 may be used to provide a normal to the table plane. If the patient is aligned with the table B, the ML axis may be in alignment with one of the axes of the table reference 40, for the normal to the table plane to be transferred between the table reference 40 and the bone digitizer 30. If the patient lies in lateral decubitus on the support table B and is aligned with table edges, the lateral axis of the support table B is used to define the AP axis of the pelvis. Accordingly, as shown in FIG. 2, the table reference 40 may be used to provide the lateral axis of the support table B. By relating the table reference 40 to the reference orientation as set forth in 63, the inertial sensor units of the table reference 40 and that of the pelvic frame of reference 50 communicate information so as to transfer the normal of the plane table (supine decubitus) or the lateral axis of the table support (lateral decubitus) to the pelvic frame of reference 50, thereby defining the AP axis of the patient. As also set forth in 63, the inertial sensor units of the caliper instrument 10 and that of the pelvic frame of reference 50 communicate information so as to transfer the X axis of the caliper instrument to the pelvic frame of reference 50, thereby defining the ML axis of the patient. A cross-product of the ML axis and of the AP axis is the longitudinal axis of the patient.

In lateral decubitus, a reference orientation can also be defined such that the table plane normal provides the patient ML axis and the table lateral axis provides the patient antero-posterior axis. In supine decubitus, a reference orientation can also be defined such that the table plane normal provides the patient antero-posterior axis and the table lateral axis provides the patient medio-lateral axis. By relating the table reference 40 to the reference orientation as set forth in 63, the inertial sensor units of the table reference 40 and that of the pelvic frame of reference 50 communicate information so as to transfer the table normal and lateral axis to the pelvic frame of reference 50, thereby defining a ML axis and an antero-posterior axis of the patient. A cross-product of the medio-lateral axis and of the antero-posterior axis is the longitudinal axis of the patient.

According to 63, the inertial sensor units communicate their relative position by rotating the support table around its lateral axis (Trendelenburg/reverse Trendelenburg), using the algorithm described in PCT international publication no. WO 2011/088541 with the table being the object of the calibration, where the two sensor units are fixed relative to each other. If using the caliper instrument 10, the sensor unit on the caliper instrument 10 can rotate around the axis between the legs 12 since only the orientation of that axis, compared to the other inertial sensor unit, is used. The algorithm used to compute the relative position between two inertial sensors device would need to be adapted to compensate for that motion.

According to 64, the surgical procedure may be performed using the frame of reference that has been defined in the previous step for bone navigation, and transferred to any appropriate pelvic reference.

1. A method for performing a surgical procedure related to a pelvis of a patient, the method comprising:
adjusting a length between ends of a digitizer device to a distance between opposite landmarks of the pelvis of the patient;
applying the ends of the digitizer device against the opposite landmarks of the pelvis;
initializing an inertial sensor unit of the digitizer device to set an orientation of the digitizer device relative to a medio-lateral axis of the patient, the medio-lateral axis of the patient being part of a pelvic coordinate system; and
navigating at least one tool within the pelvic coordinate system during a surgical procedure.

2. The method according to claim 1, further including positioning a table reference device on an operating table supporting the patient in supine decubitus, and initializing an inertial sensor unit of the table reference device to set an orientation of the table reference device relative to a support plane defined by the operating table.

3. The method according to claim 2, including setting a line parallel to the support plane of the operating table in the inertial sensor unit of the table reference device to be a longitudinal axis of the patient in supine decubitus, the longitudinal axis being part of the pelvic coordinate system

4. The method according to claim 2, including setting a normal to the support plane of the operating table in the inertial sensor unit of the table reference device to be an anterior-posterior axis of the patient in supine decubitus, the anterior-posterior axis being part of the pelvic coordinate system.

5. The method according to claim 4, comprising obtaining a third axis from the medio-lateral and anterior-posterior axes, the third axis being a longitudinal axis of the patient in supine decubitus, the longitudinal axis being part of the pelvic coordinate system.

6. The method according to claim 1, wherein adjusting the length between the ends of the digitizer device includes displacing the ends along a direction being normal to a direction of the length.

7. The method according to claim 1, wherein adjusting the length between the ends of the digitizer device or applying the ends of the digitizer device includes locking the ends of the digitizer device in position when the length between the ends is a desired length.

8. The method according to claim 1, wherein initializing the inertial sensor unit of the digitizer device includes providing the inertial sensor unit with a preset orientation, the method including mounting the inertial sensor unit onto the digitizer device, the mounting of the inertial sensor unit onto the digitizer device aligning the preset orientation with the length between the ends of the digitizer device.

9. The method according to claim 8, wherein the preset orientation of the inertial sensor unit is defined in a three-axis coordinate system, the mounting of the inertial sensor unit onto the digitizer device aligning the three-axis coordinate system of the preset orientation with three axes of the digitizer device.

10. The method according to claim 8, including resetting the inertial sensor unit after mounting the inertial sensor unit to the digitizer device to obtain the preset orientation.

11. The method according to claim 1, wherein applying the ends of the digitizer device against the opposite landmarks of the pelvis includes applying the ends against the anterior superior iliac spines (ASIS) on opposite sides of the pelvis.

12. The method according to claim 1, further including associating the pelvic coordinate system with a trackable object secured to the pelvis.
